(19) United States
(12) Patent Application Publication
(10) Pub. No.: US 2005/0194007 A1
(43) Pub. Date: Sep. 8, 2005

(54) Method and apparatus for producing diluted solutions

(76) Inventor: Hans Georg Hagleitner, Zell am See (AT)

Correspondence Address: LERNER AND GREENBERG, PA, P O BOX 2480, HOLLYWOOD, FL, 33022-2480, US

(21) Appl. No.: 11/073,903

(22) Filed: Mar. 7, 2005

(30) Foreign Application Priority Data
Mar. 5, 2004 (AT) A 378/2004

(51) Int. Cl. A61M 11/00

(52) U.S. Cl. 128/200.23

(57) ABSTRACT

In the production of diluted solutions in different concentrations, in each case an amount of a concentrate and an amount of a diluent are mixed. The ratio of the two amounts is never changed. The different concentrations of the diluted solutions result from concentrates of different concentrations that are used.

### BACKGROUND OF THE INVENTION

### FIELD OF THE INVENTION

The invention relates to a method and an apparatus for producing diluted solutions in different concentrations from concentrates, in particular from different concentrates. In each case, an amount of a concentrate and an amount of a diluent being mixed.

In the production of diluted solutions, customarily, a multiple of units of volume of the diluent is added to one unit of volume of the concentrate. The multiple corresponds to the dilution factor.

If the diluted solutions are to have different concentrations, the procedure remains the same. That is to say, for more highly diluted solutions, more units of volume of a diluent are mixed.

This, in principle, completely satisfactory procedure has disadvantages, since measurement errors can occur. These principally consist in the fact that the volume of the diluent is determined incorrectly, or the number of units of volume is calculated incorrectly.

The errors of a wrong measurement determination occur considerably more frequently than errors in the production of concentrates having different concentrations, since the amount is measured by the consumer or user, whereas the concentration is customarily set by the manufacturer of the concentrate.

Thus, for example, in cleaners or disinfectants, by adding one closure cap of the agent to one bucket of water, a constant concentration is virtually impossible, since neither the water bucket nor the closure cap are always filled with constant quantities.

In addition, there is also the effect that frequently an additional portion of the concentrate is added to ensure the action, which is not necessary, however.

### SUMMARY OF THE INVENTION

It is accordingly an object of the invention to provide a method and a device for producing diluted solutions in a variety of concentrations, which overcome the above-mentioned disadvantages of the heretofore-known devices and methods of this general type.

With the foregoing and other objects in view there is provided, in accordance with the invention, a method for producing diluted solutions in mutually different concentrations, which comprises:
providing an amount of concentrate, in particular diverse concentrates;
providing an amount of diluent;
mixing the concentrate and the diluent and thereby keeping a ratio between the amount of concentrate and the amount of diluent constant, and producing the mutually different concentrations of the diluted solutions by using concentrates having mutually different concentrations.

In accordance with an added feature of the invention, the concentrates having mutually different concentrations are produced by prediluting the concentrate, or a plurality of diverse concentrates.

In accordance with an additional feature of the invention, the amount of concentrate and the amount of diluent are kept constant by actuating two metering apparatuses having mutually different flow rates per unit time for an equal length of time. In the alternative, the amount of concentrate and the amount of diluent are kept constant by actuating two portioning apparatuses of mutually different volumes at least once.

With the above and other objects in view there is also provided, in accordance with the invention, an apparatus for producing diluted solutions in different concentrations, comprising: a first metering apparatus for metering in an amount of a concentrate; a second metering apparatus for metering in an amount of a diluent for mixing with the concentrate; the first metering apparatus having a first flow rate per unit time, and the second metering apparatus having a second flow rate per unit time different from the first flow rate.

With the above and other objects in view there is also provided, in accordance with the invention, an apparatus for producing diluted solutions in different concentrations, comprising: a first portioning apparatus for feeding an amount of a concentrate; a second portioning apparatus for feeding an amount of a diluent for mixing with the concentrate; the first portioning apparatus having a first volume, and the second portioning apparatus having a second volume different from the first volume.

In other words, the wrong dosage is avoided with this invention by keeping the ratio of the two amounts constant and producing the different concentrations of the diluted solutions by using concentrates having different concentrations. For the user this therefore produces the advantage that the two amounts of concentrate and solvent are provided in fixed sizes, and the user only needs to always combine these two amounts.

Furthermore, to produce the diluted solutions of different concentrations, simpler metering or portioning apparatuses can be used which deliver constant flow rates or portions of constant volumes.

Other features which are considered as characteristic for the invention are set forth in the appended claims.

Although the invention is illustrated and described herein as embodied in a method for the production of diluted solutions, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of a more specific exemplary embodiment.

### EXAMPLE

By way of example, one liter of a ten percent strength solution can be produced by mixing 10 centiliters (cl) of a concentrate and 90 centiliters of a diluent (i.e., diluting solution, thinner). A portioning apparatus suitable therefor has two containers of these capacities. This portioning apparatus can equally well be used to produce a five percent strength solution which must comprise 95 centiliters of diluent. In this case a half prediluted concentrate is used, since the 10 centiliters of the prediluted concentrate which is delivered by the portioning apparatus already comprise the 5 centiliters of diluent missing from the 95 centiliters.

For larger amounts, preferably, as mentioned, metering apparatuses having constant flow rates per unit time are chosen. Twenty liters of the above-mentioned ten-percent strength solution are charged in one minute by metering apparatuses having flow rates of two liters and eighteen liters per minute, respectively. Twenty liters of a five percent strength solution are likewise produced in one minute using these metering apparatuses if the concentrate is prediluted by half and already comprises one liter of diluent.

The inventive method is especially of importance since, in one and the same metering or portioning apparatus, different compositions can be diluted which are customarily not used in the same dilution. It is only necessary to offer the different compositions in identical exchangeable bottles or containers which can be inserted into the apparatus.

This application claims the priority, under 35 U.S.C. § 119, of Austrian patent application No. A 378/2004, filed Mar. 5, 2004; the entire disclosure of the prior application is herewith incorporated by reference.

1. A method for producing diluted solutions in mutually different concentrations, which comprises:
providing an amount of concentrate;
providing an amount of diluent;
mixing the concentrate and the diluent and thereby keeping a ratio between the amount of concentrate and the amount of diluent constant, and producing the mutually different concentrations of the diluted solutions by using concentrates having mutually different concentrations.

2. The method according to claim 1, which comprises producing the concentrates having mutually different concentrations by prediluting the concentrate.

3. The method according to claim 1, which comprises producing the concentrates having mutually different concentrations by prediluting a plurality of diverse concentrates.

4. The method according to claim 1, which comprises keeping the amount of concentrate and the amount of diluent constant by actuating two metering apparatuses having mutually different flow rates per unit time for an equal length of time.

5. The method according to claim 1, which comprises keeping the amount of concentrate and the amount of diluent constant by actuating two portioning apparatuses of mutually different volumes at least once.

6. An apparatus for producing diluted solutions in different concentrations, comprising:
a first metering apparatus for metering in an amount of a concentrate;
a second metering apparatus for metering in an amount of a diluent for mixing with the concentrate;
said first metering apparatus having a first flow rate per unit time, and said second metering apparatus having a second flow rate per unit time different from said first flow rate.

7. The apparatus according to claim 6, wherein said first metering apparatus is configured to meter in a plurality of diverse concentrates.

8. An apparatus for producing diluted solutions in different concentrations, comprising:
a first portioning apparatus for feeding an amount of a concentrate;
a second portioning apparatus for feeding an amount of a diluent for mixing with the concentrate;
said first portioning apparatus having a first volume, and said second portioning apparatus having a second volume different from said first volume.

9. The apparatus according to claim 6, wherein said first portioning apparatus is configured to feed in a plurality of diverse concentrates.
